(19) United States
(12) Patent Application Publication
(10) Pub. No.: US 2025/0262372 A1
(43) Pub. Date: Aug. 21, 2025

(54) REMOVABLE TIP FOR DEVICE AND METHOD FOR CLEANING NASAL CAVITIES

(71) Applicant: Kemi Azeez, Lima, OH (US)

(72) Inventors: Kemi Azeez, Lima, OH (US); David Uhlenhake, Toledo, OH (US)

(21) Appl. No.: 19/057,252

(22) Filed: Feb. 19, 2025

Related U.S. Application Data
(60) Provisional application No. 63/555,331, filed on Feb. 19, 2024.

(51) Int. Cl. A61M 3/02

(52) U.S. Cl. CPC A61M 3/0279; A61M 2205/14; A61M 2205/3327; A61M 2210/0618

(57) ABSTRACT

A removable tip for a nasal cleaning device includes an applicator body having a predetermined shape formed from a compressible material. The removable tip may be configured to be applied to a nasal cavity of a user. The removable tip may also include a hollow tube partially disposed in the applicator body. The hollow tube may have a first opening at a top end disposed in the applicator body and a second opening at a bottom end. The bottom end may be configured to be removably secured to the nasal cleaning device, selectively placing the hollow tube in fluid communication with a delivery tube configured to deliver a volume of saline solution from a clean reservoir of the nasal cleaning device to the hollow tube and the applicator body of the removable tip.

### CROSS-REFERENCE TO RELATED APPLICATIONS

This application claims the benefit of U.S. Provisional Application No. 63/555,331, filed on Feb. 19, 2024. The entire disclosure of the above application is incorporated herein by reference.

### FIELD

The present technology relates to cleaning a nasal cavity and, more particularly, to a removable tip for a nasal cleaning device and methods for selecting and using a removable tip.

### INTRODUCTION

This section provides background information related to the present disclosure which is not necessarily prior art.

The field of personal healthcare devices has seen significant advancements in recent years, particularly in the development of devices designed for cleaning nasal cavities. Nasal cleaning maintains nasal hygiene by removing mucus, dust, bacteria, viruses, and fungi that accumulate in the nasal passages. Methods for nasal cleaning include sinus flushing using a saline solution, manual scraping, and sponging, However, these methods can be uncomfortable, unsanitary, messy, and often provide incomplete cleaning. Devices used for such methods can furthermore be difficult to clean, complicated to use, and expensive.

Recent developments in the field have led to nasal cleaning devices that are easier to use, sanitary, and provide a improved cleaning of the nasal cavity. One such device is described in the Applicant's co-pending U.S. patent application Ser. No. 18/477,669, filed on Sep. 29, 2023, the entire disclosure of which is incorporated herein by reference. This device includes a housing with a head assembly and a fluid tank. The head assembly includes a rotatable tip and a basin that circumscribes the tip. The fluid tank is divided into clean and waste reservoirs. This device provides an improved method for nasal cleaning by allowing for a more thorough and comfortable cleaning of the nasal cavity.

The rotatable tip of the head assembly in Applicant's co-pending U.S. patent application Ser. No. 18/477,669 is designed to oscillate and/or rotate, providing a comprehensive cleaning of the nasal cavity. The tip is removable and may be easily replaced or sanitized after each use. This feature is particularly beneficial for maintaining hygiene. Additionally, the removable tip may be removed and replaced by a different removable tip to accommodate nasal cavities having variable sizes and shapes, and to accommodate user preferences. The basin is configured to catch solid and liquid waste, thereby militating against messy contact with the user during operation.

Despite such improvements, nasal cleaning devices continue to present certain limitations in the form and function of the removable tips. Removable tips, while functional, lack customizable features in terms of size, shape, and material, which can limit the effectiveness of the removable tips and the comfort of the removable tip depending on the user. Certain removable tips also do not provide a consistent, reliable cleaning of the nasal cavity. Another concern relates to counterfeit tips, which can compromise the performance, hygiene, and safety with respect to operation of the nasal cleaning device and performance thereof.

Accordingly, there is a continuing need for a removable tip for a nasal cleaning device that provides more personalized, effective, and hygienic cleaning. Desirably, the removable tip would include a unique form and function to better match the contours of various nasal cavities. It is also desirable to minimize a risk of using inauthentic components, thereby ensuring users have access to safe and sanitary removable tips for nasal cleaning.

### SUMMARY

In concordance with the instant disclosure, a removable tip for a nasal cleaning device that provides a more personalized, effective, and hygienic cleaning, includes a unique form and function to better match the contours of various nasal cavities, and minimizes a risk of using an inauthentic component, thereby ensuring a user has access to a safe and sanitary removable tip for nasal cleaning, has surprisingly been discovered. The present technology includes articles of manufacture, systems, and processes that relate to a removable tip for a nasal cleaning device that offers enhanced comfort, cleaning, and customization.

In certain embodiments, a removable tip for a nasal cleaning device may include an applicator body having a predetermined shape formed from a compressible material. The removable tip may be configured to be applied to a nasal cavity of a user. The removable tip may also include a hollow tube partially disposed in the applicator body. The hollow tube may have a first opening at a top end disposed in the applicator body and a second opening at a bottom end. The bottom end may be configured to be removably secured to the nasal cleaning device, selectively placing the hollow tube in fluid communication with a delivery tube configured to deliver a volume of saline solution from a clean reservoir of the nasal cleaning device to the hollow tube and the applicator body of the removable tip.

In certain embodiments, a nasal cleaning device includes a housing and a head assembly coupled to the housing. The head assembly may include a tip driver and a basin. A clean reservoir may be disposed in the housing. A delivery tube may be in fluid communication with the clean reservoir. The nasal cleaning device may further include a removable tip having an applicator body with a predetermined shape formed from a compressible material. The removable tip may be configured to be applied to a nasal cavity of a user. The removable tip may also include a hollow tube partially disposed in the applicator body. The hollow tube may have a first opening at a top end disposed in the applicator body and a second opening at a bottom end. The bottom end may be configured to be removably secured to the nasal cleaning device, selectively placing the hollow tube in fluid communication with a delivery tube configured to deliver a volume of saline solution from the clean reservoir of the nasal cleaning device to the hollow tube and the applicator body of the removable tip. The bottom end of the hollow tube of the removable tip may be removably secured to the head assembly.

In certain embodiments, a kit for a nasal cleaning device includes an assortment of removable tips. Each removable tip includes an applicator body having a predetermined shape formed from a compressible material. The removable tip may be configured to be applied to a nasal cavity of a user. The removable tip may also include a hollow tube partially disposed in the applicator body. The hollow tube may have a first opening at a top end disposed in the applicator body and a second opening at a bottom end. The bottom end may be configured to be removably secured to the nasal cleaning device, selectively placing the hollow tube in fluid communication with a delivery tube configured to deliver a volume of saline solution from a clean reservoir of the nasal cleaning device to the hollow tube and the applicator body of the removable tip. The assortment of removable tips may include a plurality of sizes and compressible materials to accommodate different nasal cavity dimensions and user preferences. Additionally, a nasal cavity measuring tool and instructions for selecting a removable tip based on a measured nasal cavity size may be included.

In certain embodiments, a method of selecting and using a removable tip and using the removable tip for cleaning a nasal cavity may include a first step of selecting a removable tip based on a nasal cavity of the user and user preferences. The removable tip may include an applicator body having a predetermined shape formed from a compressible material. The removable tip may be configured to be applied to a nasal cavity of a user. The removable tip may also include a hollow tube partially disposed in the applicator body. The hollow tube may have a first opening at a top end disposed in the applicator body and a second opening at a bottom end. The bottom end may be configured to be removably secured to the nasal cleaning device, selectively placing the hollow tube in fluid communication with a delivery tube configured to deliver a volume of saline solution from a clean reservoir of the nasal cleaning device to the hollow tube and the applicator body of the removable tip. A second step may include coupling the bottom end of the hollow tube to the nasal cleaning device. In a third step, the nasal cleaning device may be activated to deliver the volume of saline solution from the clean reservoir to the applicator body of the removable tip. A fourth step may include inserting the applicator body of the removable tip into the nasal cavity of the user, and a fifth step may include operating the nasal cleaning device to clean the nasal cavity.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a side elevational view of a nasal cleaning device with a removable tip, according to one embodiment.

FIG. 2 is an enlarged, fragmentary, top perspective view of a portion of a nasal cleaning device with a removable tip, according to one embodiment.

FIG. 3 is a cross-sectional side elevational view of a portion of a nasal cleaning device with a removable tip, according to one embodiment.

FIG. 4 is an exploded, fragmentary perspective view of a portion of a nasal cleaning device with a removable tip, according to one embodiment.

FIG. 5 is an exploded, cross-sectional side elevational view of a removable tip, according to one embodiment.

FIGS. 6A-6E show a perspective view (FIG. 6A), a side elevational view (FIG. 6B), a top plan view (FIG. 6C), a bottom plan view (FIG. 6D), and a cross-sectional side elevational view (FIG. 6E) of a removable tip, according to one embodiment.

FIGS. 7A-7E show a perspective view (FIG. 7A), a side elevational view (FIG. 7B), a top plan view (FIG. 7C), a bottom plan view (FIG. 7D), and a cross-sectional side elevational view (FIG. 7E) of a removable tip, according to one embodiment.

FIGS. 8A-8E depict a perspective view (FIG. 8A), a side elevational view (FIG. 8B), a top plan view (FIG. 8C), a bottom plan view (FIG. 8D), and a cross-sectional side elevational view (FIG. 8E) of a removable tip, according to one embodiment.

FIGS. 9A-9E show a perspective view (FIG. 9A), a side elevational view (FIG. 9B), a top plan view (FIG. 9C), a bottom plan view (FIG. 9D), and a cross-sectional side elevational view (FIG. 9E) of a removable tip, according to one embodiment.

FIGS. 10A-10E depict a perspective view (FIG. 10A), a side elevational view (FIG. 10B), a top plan view (FIG. 10C), a bottom plan view (FIG. 10D), and a cross-sectional side elevational view (FIG. 10E) of a removable tip, according to one embodiment.

FIG. 11 is a block diagram illustrating certain aspects of a removable tip, according to one embodiment.

FIG. 12 depicts a block diagram illustrating certain aspects of a removable tip, according to one embodiment.

FIG. 13 is a block diagram illustrating certain aspects of a removable tip, according to one embodiment.

FIG. 14 illustrates a block diagram illustrating certain aspects of a removable tip, according to one embodiment.

FIG. 15 is a block diagram illustrating certain aspects of a nasal cleaning device, according to one embodiment.

FIG. 16 is a block diagram illustrating certain aspects of a kit, according to one embodiment.

FIG. 17 is a flowchart illustrating a method of selecting a suitable removable tip, according to one embodiment.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Applicant's co-pending U.S. patent application Ser. No. 18/477,669, filed on Sep. 29, 2023 and titled CLEANING DEVICE AND METHOD FOR NASAL CAVITIES, is incorporated herein by reference. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

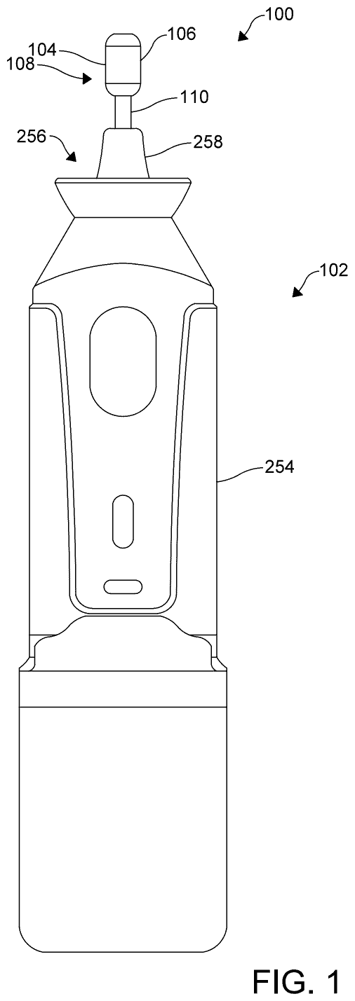

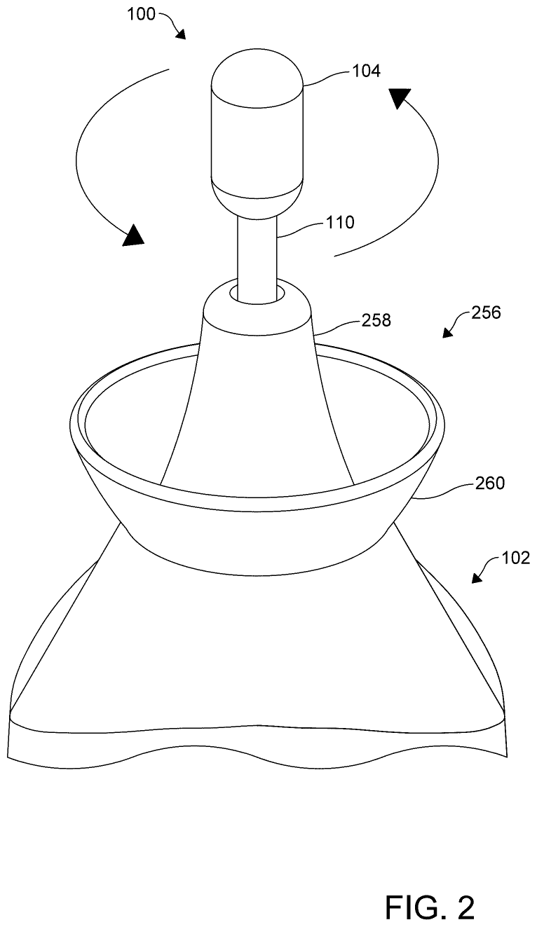

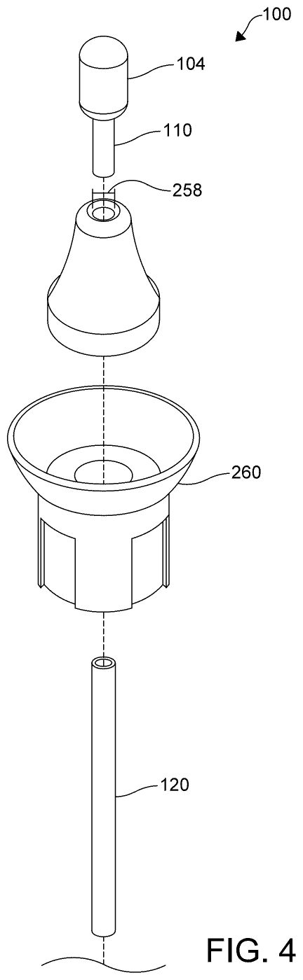

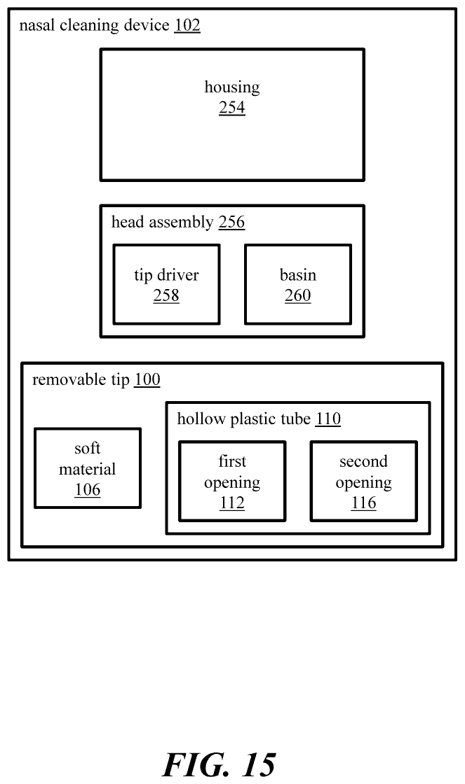

The present technology, as shown in FIGS. 1-17, improves upon existing devices and methods for nasal cleaning by introducing a removable tip 100 configured to conform to unique contours of nasal cavities, providing a more personalized, comfortable, and effective nasal cleaning. The removable tip 100 may be configured to provide a gentle yet thorough cleaning that may help alleviate symptoms associated with day-to-day use, nasal congestion, allergies, and post-operative care. Additionally, the present technology may include features for authenticating the removable tip 100 to ensure compatibility with an associated device, as well as a variety of removable tip configurations, softness and/or compressibility levels, and saturation levels to cater to a variety of personalized user preferences and needs.

As shown in FIGS. 1-5 and 11-14, the removable tip 100 may be configured for use with a nasal cleaning device 102. The removable tip 100 may include an applicator body 104 formed from a soft material 106 and having a predetermined shape 108 that is configured to be applied to a nasal cavity of a user. The removable tip 100 may also include a hollow tube 110 at least partially disposed in the applicator body 104.

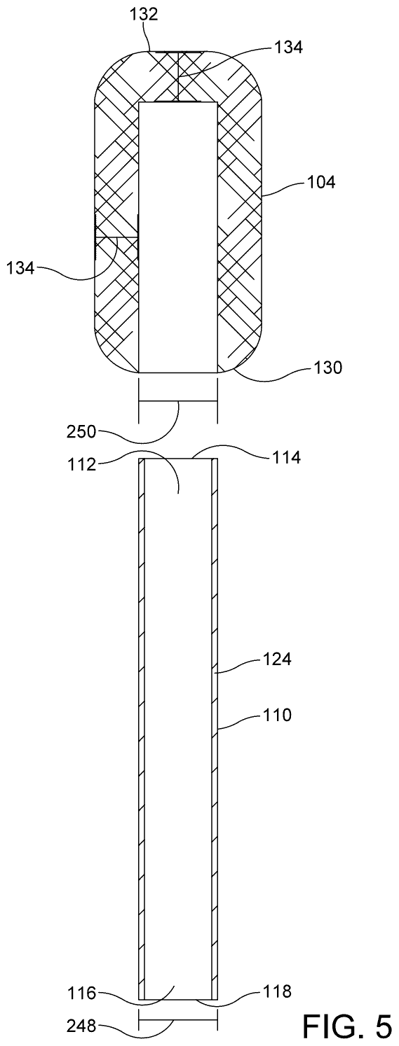

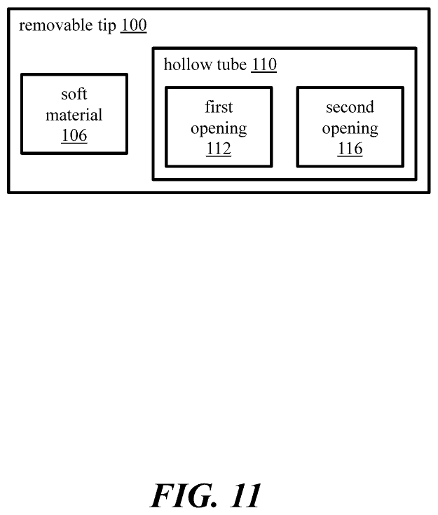

With reference now to FIGS. 5 and 11, the hollow tube 110 may have a first opening 112 at a top end 114 of the hollow tube 110 that is at least disposed in the applicator body 104 and a second opening 116 at a bottom end 118 of the hollow tube 110. The bottom end 118 of the hollow tube 110 may be configured to removably coupled to the nasal cleaning device 102. The second opening 116 at the bottom end 118 of the hollow tube 110 may selectively place the hollow tube 110 in fluid communication with a delivery tube 120 housed in the nasal cleaning device 102. The delivery tube 120 may be configured to deliver a volume of saline solution 122 from a clean reservoir 257 housed in the nasal cleaning device 102 to the hollow tube 110.

The hollow tube 110 may include or be formed using medical-grade plastics such as polyethylene, polypropylene, polycarbonate, or any other materials that are durable, flexible, hygienic, gentle, and otherwise suitable for medical applications. In certain embodiments, the hollow tube 110 may be fabricated using a polyvinyl chloride material or any other suitable material configured to form a hard, thin tube. The hollow tube 110 may include a flexible material such as biocompatible silicone to enhance comfort during use. In certain embodiments, a more rigid structure may be desirable, and material or combination of materials such acrylic and/or certain grades of stainless steel that are corrosion-resistant and easily sterilized may be utilized. The hollow tube 110 may be fabricated using any suitable combination of materials, such as a rigid plastic body having a soft silicone tip or sleeve, as examples, thereby combining the benefits of structural integrity and comfort. The hollow tube 110 may be fabricated using a transparent or semi-transparent material to allow the user to see a flow of the saline solution 122 therethrough, thereby enhancing the usability of the nasal cleaning device 102. Biodegradable plastics derived from natural sources such as corn starch may also be utilized for the hollow tube 110. The biodegradable plastics may be configured to break down more easily after use and disposal, thereby reducing the environmental impact of the removable tip 100.

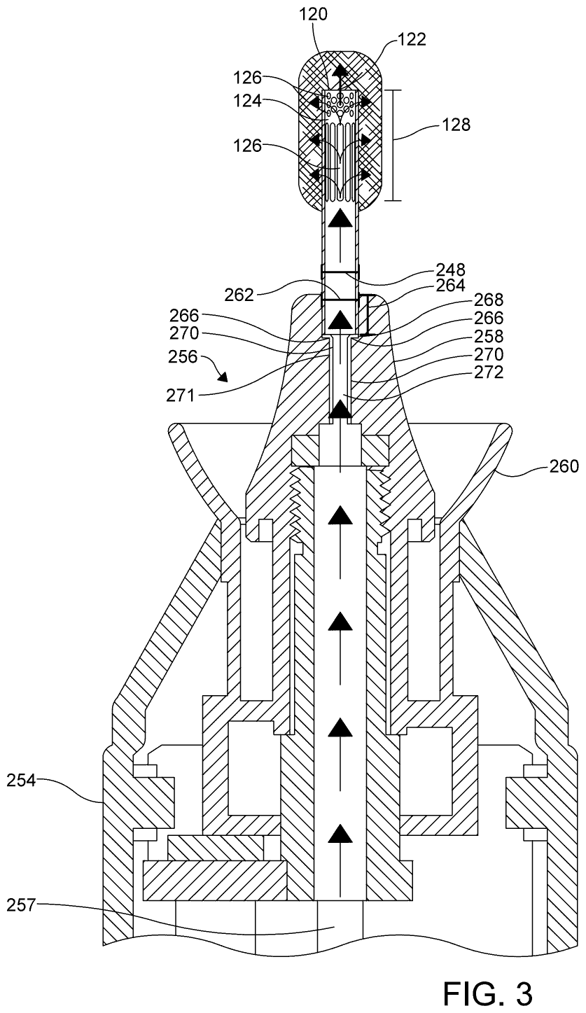

As shown in FIG. 3, a hollow tube sidewall 124 may have one or more apertures 126 disposed therein and configured to release the saline solution 122 directly onto the applicator body 104 and/or into the nasal cavity of the user. The apertures 126 may be formed at various locations along a length 128 of the hollow tube sidewall 124 that is disposed in the applicator body 104. It should be appreciated that areas of the hollow tube sidewall 124 that are not disposed in the applicator body 104 may not include apertures 126, thereby militating against saline solution 122 flowing out of an area that is not adjacent the applicator body 104. The saline solution 122 may flow out of the apertures 126 as the saline solution flows from the bottom end 118 of the hollow tube 110 to the top end 114 of the hollow tube 110. The apertures 126 may be disposed in a pattern formation or randomly. It should be appreciated that any suitable number, shape, size, and configuration of apertures 126 may be disposed in the hollow tube sidewall 124 at any desired position or plurality of positions along the length 128 of the hollow tube 110.

In certain embodiments, a selection of hollow tubes 110 having a variety of formations and configurations with respect to the number, shape, size, and configuration of the apertures 126, may be available to the user and allow the user to select a personalized hollow tube 110 having a desired flow pattern and nasal flush pressure. For example, the user may select a hollow tube 110 including a plurality of apertures 126 such that the desired flow pattern includes a flow of saline solution 122 through each of the apertures 126 and the first opening of the hollow tube 110. Alternatively, the user may select a hollow tube 110 having no apertures 126 such that the desired flow pattern includes a flow of saline solution through only the first opening 112, thereby increasing the nasal flush pressure at the first opening 112 of the hollow tube 110.

As shown in FIGS. 1-5, the applicator body 104 may include a first end 130 configured to couple to the top end 114 of the hollow tube 110 and a second end 132 positioned opposite the first end 130. The applicator body 104 may be fabricated using any suitable material that is durable, hygienic, gentle, and otherwise suitable for medical applications, such as cotton, foam, synthetic fabric, sponge material, or combinations thereof. The synthetic fabric may be a non-woven material designed for single use, and the sponge material may be a hydrophilic polyurethane foam, as examples. The material may be fibrous, porous, and/or may have air space. In certain embodiments, the material may be compressible. The compressible material may be configured to change shape when pressed against the contours in a nasal cavity during use. The material may be flexible and/or elastic, as determined by one of skill in the art.

The applicator body 104 may be available in a variety of textures ranging from soft to firm to accommodate different user preferences and sensitivity levels. A level of softness may be categorized into at least three distinct levels: soft, medium, and firm, thereby providing options for the user and enhancing the comfort, intensity, and effectiveness of the nasal cleaning. Each level of softness may be achieved by using a predetermined density and compressibility of the applicator body 104.

Examples of testable parameters for selecting and determining softness levels of the applicator body 104 may include Shore durometer readings for measuring resistance to indentation and providing a quantifiable scale of hardness with respect to the applicator body 104. For example, a lower Shore A durometer score may indicate a softer material suitable for categorization as soft, while a higher score may correspond to categorization as firm. Compression testing may also be employed. More specifically, the degree to which the applicator body 104 compresses under a predetermined load may be measured, and greater compressibility may be categorized as soft. Tensile strength tests may be used to assess how the applicator body 104 performs when stretched, with softer materials exhibiting lower tensile strength. Additionally, user feedback studies may be conducted to correlate subjective perceptions of softness with objective measurements, thereby ensuring that the quantifiable levels of softness align with user comfort and expectations. Parameters such as resistance to indentation, hardness, compressibility, and tensile strength, as examples, may improve a consistency of production and quality control of the removable tip 100, ensuring that each applicator body 104 meets a predetermined criteria for user comfort and nasal cleaning efficacy.

The applicator body 104 of the removable tip 100 may be available in a plurality of sizes and configurations and adapted to conform to nasal cavities having different shapes and sizes. In certain embodiments, the applicator body 104 may be available in sizes ranging from approximately 6 mm to 9 mm in diameter and have a length ranging from approximately 15 mm to 25 mm for pediatric use. The applicator body 104 may also be available in sizes ranging from approximately 9 mm to 14 mm in diameter and have a length ranging from approximately 25 mm to 35 mm for adult use. Other suitable dimensions may also be selected. It should be appreciated that the applicator body 104 may be any suitable length, width, height, diameter, and/or other dimensions, as desired, configured to allow the nasal cavity to be effectively cleaned, as determined by one of skill in the art. In certain embodiments, the applicator body 104 may be color-coded based on size and/or configuration to facilitate easy identification and selection of a desired size and/or shape by the user.

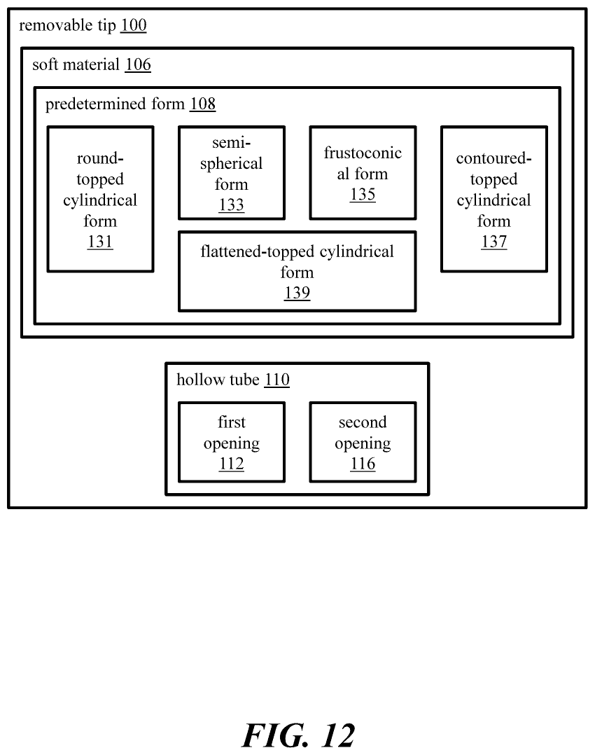

As shown in FIG. 12, the predetermined shape 108 of the applicator body 104 may be any suitable shape, with examples including a round-topped cylindrical form 131 with a rounded top and bottom, a semi-spherical form 133, a frustoconical form 135, a contoured-topped cylindrical form 137 with a contoured top and bottom, and/or a flattened-topped cylindrical form 139. Each predetermined shape 108 may facilitate easy insertion and removal from the nasal cavity while maximizing contact with nasal passage walls. The predetermined shape 108 for the applicator body 104 of the removable tip 100 may also include an oval-shaped form that mirrors the natural shape of the nasal passages, a bulbous form that provides a gentle massaging effect, and/or a tapered form that allows for gradual expansion to fit snugly within the nasal cavity. Additionally, the predetermined shape 108 may include additional features such as a spiral or ribbed element to enhance nasal cleaning through increased surface area and texture, thereby aiding in the removal of debris and mucus. A dual-lobed form may be employed and configured to simultaneously clean an upper nasal passage and a lower nasal passage with a single insertion.

The applicator body 104 may be configured to receive the saline solution 122 as the saline solution 122 flows out from one or more of the first opening 112 of the hollow tube 110 and/or one or more apertures 126 formed in the hollow tube sidewall 124. A level and/or pattern of saturation of the applicator body 104 and/or a section of the applicator body 104 may be determined by one or more of a volume of saline solution 122, a number of apertures 126, and an applicator body thickness 134. In certain embodiments, the applicator body thickness 134 may be measured as any width extending from an outer surface of the hollow tube 110 to an outer surface of the applicator body 104. The applicator body thickness 134 may be configured to optimize saturation of the applicator body 104, militate against a flow of saline solution 122 out of the nasal cavity during use, and/or to effectively flush and clean the nasal cavity.

In certain embodiments, the applicator body thickness 134 of the first end 130 of the applicator body 104 adjacent the hollow tube sidewall 124 may be greater than the applicator body thickness 134 of the second end 132 of the applicator body 104 adjacent the top end 114 of the hollow tube 110. As such, a nasal flush pressure may be greater at the second end 132 of the applicator body 104 due to a decreased applicator body thickness 134. A flow pattern of the saline solution 122 may include a faster flow at the second end 132 of the applicator body 104 due to the decreased applicator body thickness 134 followed by a slower flow at the first end 130 of the applicator body 104 due to an increased applicator body thickness 134. The slower flow at the first end 130 of the applicator body 104 may militate against the saline solution 122 flowing out of the nasal cavity during use.

It should be appreciated that the applicator body thickness 134 may be consistent throughout the applicator body 104 or may vary. It should be further appreciated that, in certain embodiments, the applicator body thickness 134 at the second end 132 of the applicator body 104 may have any suitable thickness and configuration for allowing a user to comfortably use the removable tip 100 without injury from contact with the top end 114 of the hollow tube 110. The applicator body thickness 134 at the second end 132 of the applicator body 104 may be further configured to ensure that the nasal flush pressure of the saline solution 122 flowing out from the second opening 116 of the hollow tube 110 allows for a sufficient nasal flush pressure without causing injury and/or discomfort to the user.

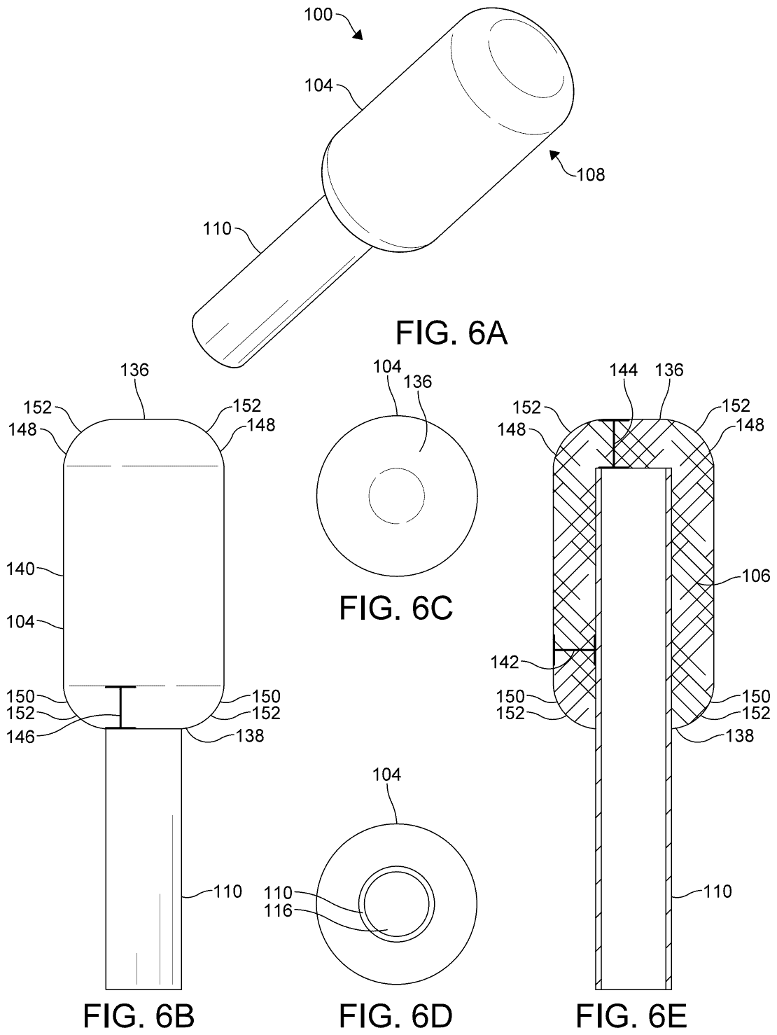

In certain embodiments, as shown in FIGS. 6A-6E, the applicator body 104 may have a predetermined shape 108 including a convex or dome shaped top portion 136, a convex or dome shaped bottom portion 138, and an elongated sidewall portion 140. A sidewall portion thickness 142, a top portion thickness 144, and a bottom portion thickness 146 may be substantially the same. Each of a first transition area 148 between the sidewall portion 140 and the top portion 136 and a second transition area 150 between the sidewall portion 140 and the bottom portion 138 may include an outer surface 152 having a radius of curvature between 1.0 mm and 2.5 mm. More particularly, the radius of curvature may be between 1.25 mm and 2.25 mm, and most particularly, the radius of curvature may be between 1.5 mm and 2.0 mm. It should be appreciated that the radius of curvature for the first transition area 148 and the second transition area 150 may be the same or different.

Advantageously, the uniform thickness of the top portion 126, the bottom portion 138, and the sidewall portion 140 may allow for an even distribution of the saline solution 122 during use, thereby providing a consistent, thorough, effective cleaning of the nasal cavity. The radius of curvature of the first transition area 148 may be configured to optimally pair with typical contours and passages in the nasal cavity to enhance the nasal cleaning. Additionally the radius of curvature of the second transition area 150 may optimally release the saline solution 120 directly into the basin during use, thereby minimizing spillage. The elongated sidewall portion 140 may allow for an effective nasal cleaning along a length of an entire nasal passage. Moreover, the elongated sidewall portion 140 may allow for an effective nasal cleaning for users having a longer and/or narrower nasal cavity.

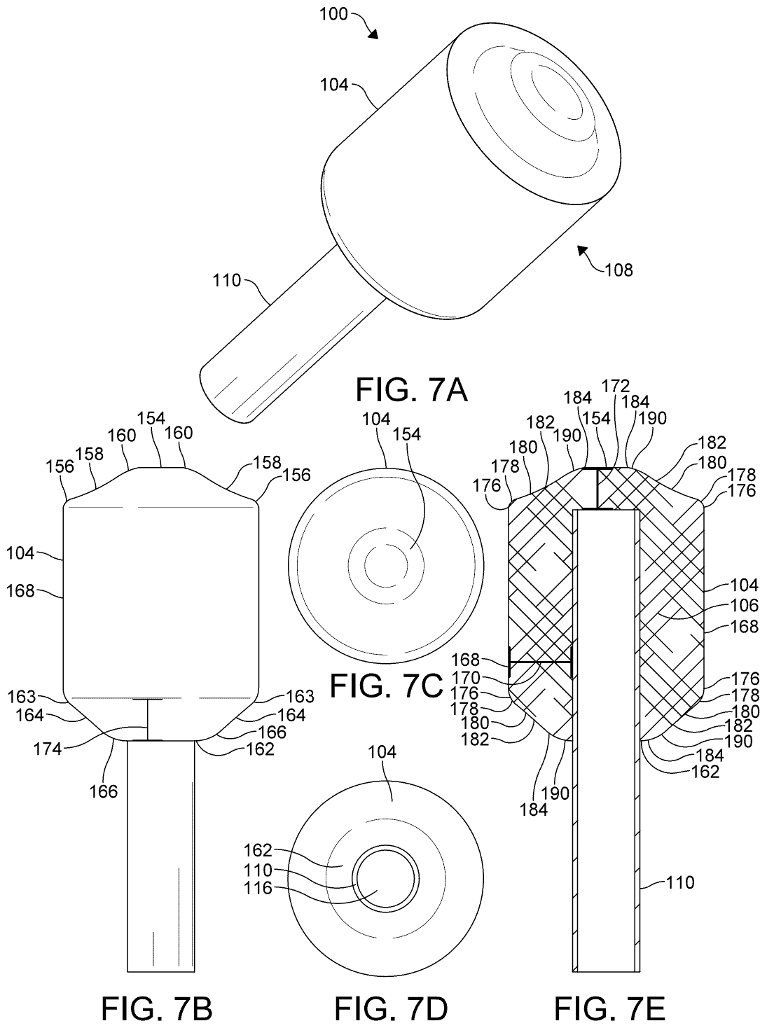

In certain embodiments, as shown in FIGS. 7A-7E, the applicator body 104 may have a predetermined shape 108 including a top portion 154 having each of a first top convex portion 156, a top concave portion 158, and a second top convex portion 160, a bottom portion 162 having each of a first bottom convex portion 163, a bottom concave portion 164, and a second bottom convex portion 166, and a sidewall portion 168. A sidewall portion thickness 170, a top portion thickness 172, and a bottom portion thickness 174 may be substantially the same, different, or variable.

A first transition area 176 between the sidewall portion 168 and each of the top concave portion 158 and the bottom concave portion 164 may include a first outer surface 178 having a radius of curvature between 0.5 mm and 1.0 mm. More particularly, the radius of curvature may be between 0.6 mm and 0.9 mm, and most particularly, the radius of curvature may be between 0.7 mm and 0.8 mm. A second transition area 180 between the first top convex portion 156 and the second top convex portion 160 and the first bottom convex portion 163 and the second bottom convex portion 166 may include a second outer surface 182 having a radius of curvature between 1.0 mm and 2.5 mm. More particularly, the radius of curvature may be between 1.25 mm and 2.25 mm, and most particularly, the radius of curvature may be between 1.5 mm and 2.0 mm. A third transition 184 area between each of the top concave portion 158 and a top surface 186 and the bottom concave portion 164 and a bottom surface 188 may include a third outer surface 190 having a radius of curvature between 0.5 mm and 1.0 mm. More particularly, the radius of curvature may be between 0.6 mm and 0.9 mm, and most particularly, the radius of curvature may be between 0.7 mm and 0.8 mm. Advantageously, the variable thickness of the applicator body 104, according to certain embodiments, may allow for an enhanced nasal flow pressure at the top portion 154 and the sidewall portion 168 without using excessive soft material 106 in areas of the applicator body 104 that do not directly receive a flow of saline solution 122 from the hollow tube 110. Additionally, the top concave portion 158 in combination with the second top convex portion 160 may allow for an enhanced nasal cleaning of more narrow, hard to reach areas of the nasal cavity.

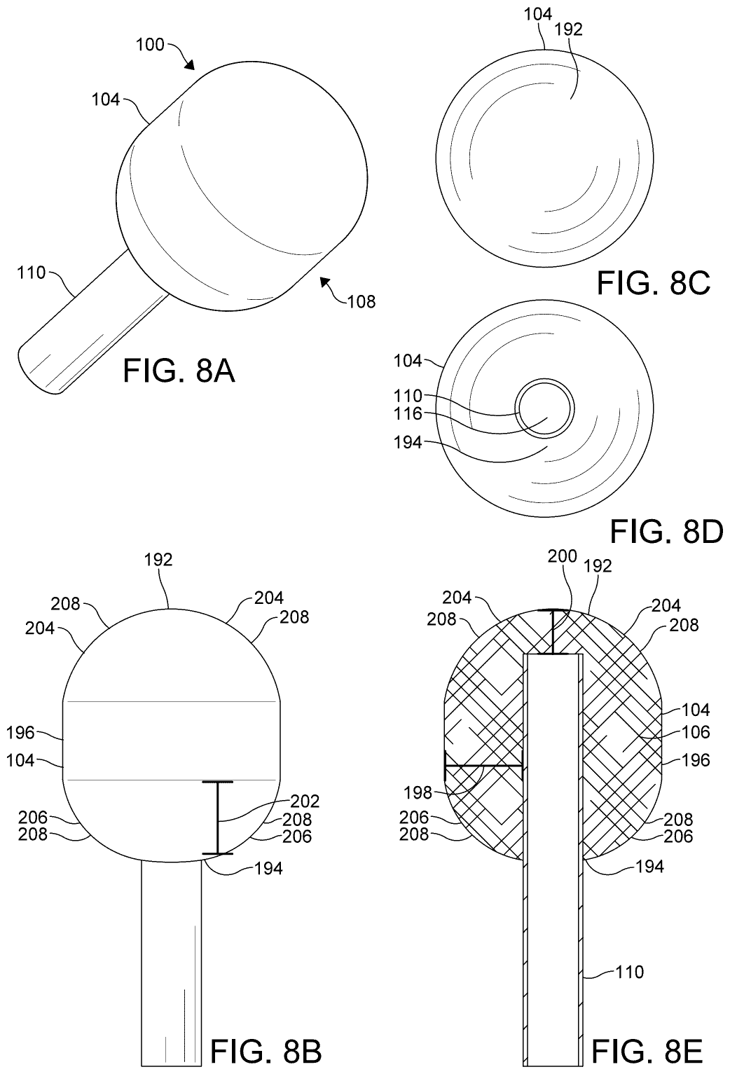

With reference now to FIGS. 8A-8E, the applicator body 104 may have a substantially spherical predetermined shape 108 including a convex or dome shaped top portion 192, a convex or dome shaped bottom portion 194, and a sidewall portion 196, according to certain embodiments. A sidewall portion thickness 198 may be greater than a top portion thickness 200. Each of a first transition area 204 between the sidewall portion 196 and the top portion 192 and a second transition area 206 between the sidewall portion 196 and the bottom portion 194 may include an outer surface 208 having a radius of curvature between 2.5 mm and 4.5 mm. More particularly, the radius of curvature may be between 3.0 mm and 4.0 mm, and most particularly, the radius of curvature may be between 3.25 mm and 3.75 mm. It should be appreciated that the radius of curvature of the first transition area 204 and the second transition area 206 may be substantially the same or different. Advantageously, the decreased thickness of the top portion thickness 200 may increase the nasal flush pressure, and the increased thickness of the sidewall portion thickness 198 may allow for increased absorption of the saline solution 122 during use and effective nasal cleaning for users having a larger and/or wider nasal cavity. A bottom portion thickness 202 may have any suitable thickness, as determined by one of skill in the art.

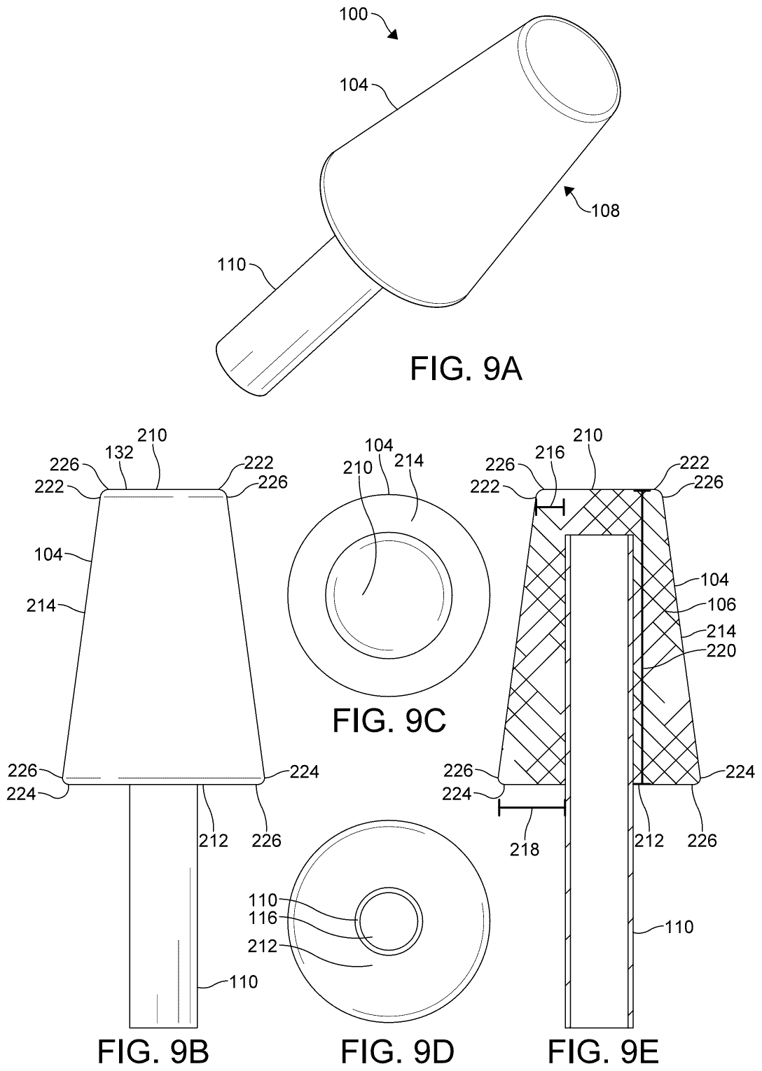

In certain embodiments, as shown in FIGS. 9A-9E, the applicator body 104 may have a substantially frustoconical predetermined shape 108 including a top portion 210, a bottom portion 212, and a sidewall portion 214. The sidewall portion 214 may have a first sidewall thickness 216 adjacent the top portion 210 and a second sidewall thickness 218 adjacent the bottom portion 212 that is greater than the first thickness. The sidewall thickness may gradually increase along a length 220 of the applicator body 104 extending from the first sidewall thickness 216 to the second sidewall thickness 218.

Each of a first transition area 222 between the sidewall portion 214 and the top portion 210 and a second transition area 224 between the sidewall portion 214 and the bottom portion 212 may include an outer surface 226 having a radius of curvature. The radius of curvature of the first transition area 222 may be between 0.5 mm and 2.0 mm. More particularly, the radius of curvature may be between 0.75 mm and 1.75 mm, and most particularly, the radius of curvature may be between 1.0 mm and 1.5 mm. The radius of curvature of the second transition area 224 may be between 0.5 mm and 2.0 mm. More particularly, the radius of curvature may be between 0.75 mm and 1.75 mm, and most particularly, the radius of curvature may be between 1.0 mm and 1.5 mm. It should be appreciated that the substantially frustoconical predetermined shape 108 may allow the second end 132 of the applicator body 104 to extend further into the nasal cavity, and the gradual increase of the sidewall thickness may improve absorption and prevent saline solution 122 from dripping out of the nasal cavity during use.

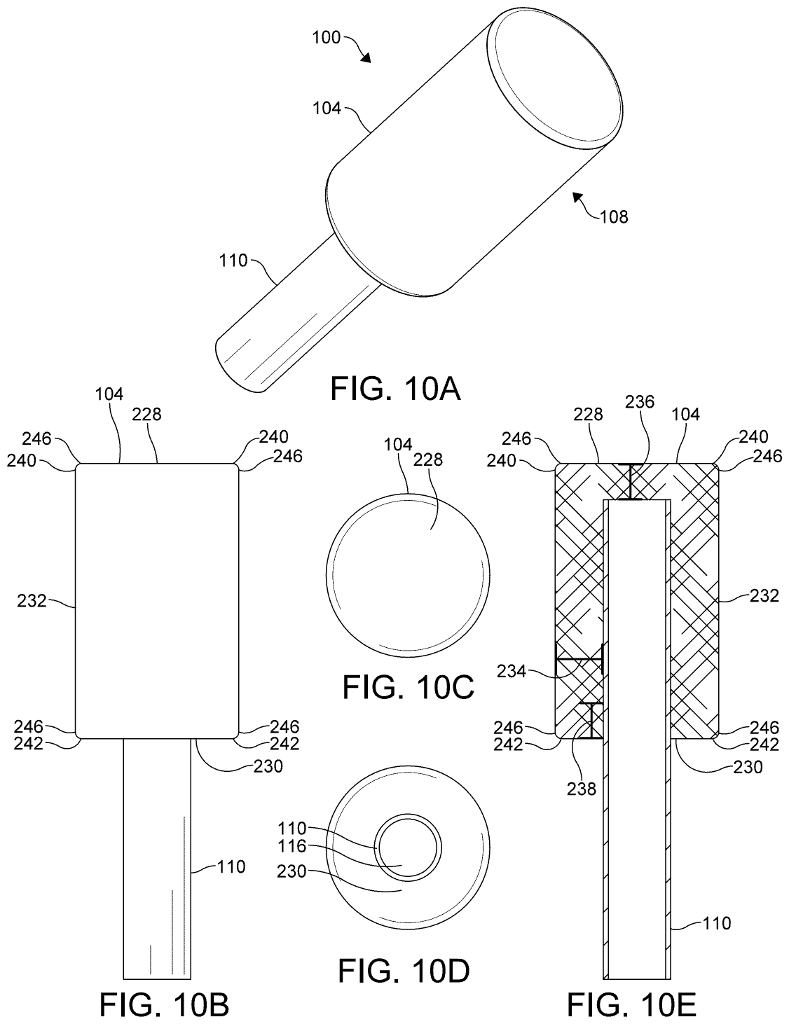

With reference now to FIGS. 10A-10E, certain embodiments may include an applicator body 104 having a substantially cylindrical predetermined shape 108 including a top portion 228, a bottom portion 230, and a sidewall portion 232. A sidewall portion thickness 234, a top portion thickness 236, and a bottom portion thickness 238 may be substantially the same or different. Each of a first transition area 240 between the sidewall portion 232 and the top portion 228 and a second transition area 242 between the sidewall portion 232 and the bottom portion 230 may include an outer surface 246 having a radius of curvature between 0.5 mm and 2.0 mm. More particularly, the radius of curvature may be between 0.75 mm and 1.75 mm, and most particularly, the radius of curvature may be between 1.0 mm and 1.5 mm. It should be appreciated that the radius of curvature of the first transition area 240 and the second transition area 242 may be the same or different. Advantageously, the substantially cylindrical predetermined shape 108 may allow a user having a more sensitive nasal cavity to use the removable tip 100 free of concern that the removable tip 100 may extend too far into the nasal cavity and cause injury. Additionally, the radius of curvature may increase a level of force the user may use apply to the nasal cavity during use.

Each predetermined shape 108 may be configured for an enhanced nasal cleaning, to improve ease of use, and to minimize discomfort during the nasal cleaning. A skilled artisan may employ any predetermined shape 108, as desired. It should be appreciated that the applicator body 104 may have any desired predetermined shape and configuration, including an amorphous shape and configuration, as determined by one of skill in the art.

The applicator body 104 may be affixed to the hollow tube 110 using a mechanical means such as a friction fit or affixed using an adhesive that is resistant to dissolution upon exposure to saline solution 122. The adhesive may include a medical-grade cyanoacrylate or a biocompatible epoxy that maintains a strong bond between the applicator body 104 and the hollow tube 110 during use, as an example. The adhesive may include a biodegradable material such as polyvinyl alcohol. The adhesive may include a desirable fragrance. Other suitable types of adhesives may also be employed, as desired.

In certain embodiments, the adhesive may form a watertight seal between the applicator body 104 and the hollow tube 110 to prevent leakage of saline solution 122. The adhesive may be applied in a pattern that allows for even distribution of saline solution 122 through the applicator body 104 from the hollow tube 110. The adhesive may be applied to any area adjacent to one or more apertures 126 formed in the hollow tube sidewall 124 such that saline solution 122 may easily flow out of the apertures 126 and onto the applicator body 104. The adhesive may be capable of withstanding mechanical forces applied during rotation and oscillation of the removable tip 100 within the nasal cavity. The adhesive may maintain its bonding properties over a wide range of temperatures, ensuring the integrity of the removable tip 100 during storage and use. The adhesive may be selected to be compatible with the materials of both the applicator body 104 and the hollow tube 110, thereby optimizing the connection between the applicator body 104 and the hollow tube 110.

In certain embodiments, where the applicator body 104 is secured to the hollow tube 110 using an interference or friction fit, the hollow tube 110 may have an outer wall diameter 248 that is substantially the same as a bore 250 formed in the applicator body 104, as shown in FIGS. 3-5. As such, a secure connection may be formed when the hollow tube 110 is forced or otherwise inserted into the bore 250 of the applicator body 104. Any suitable outer wall diameter 248 configured to form a secure friction fit with the bore 250 of the applicator body 104 may be used. Likewise, any suitable length 128 of the hollow tube sidewall 124 disposed in the applicator body 104 may be employed to form a secure connection and allow for optimal saturation of the applicator body 104.

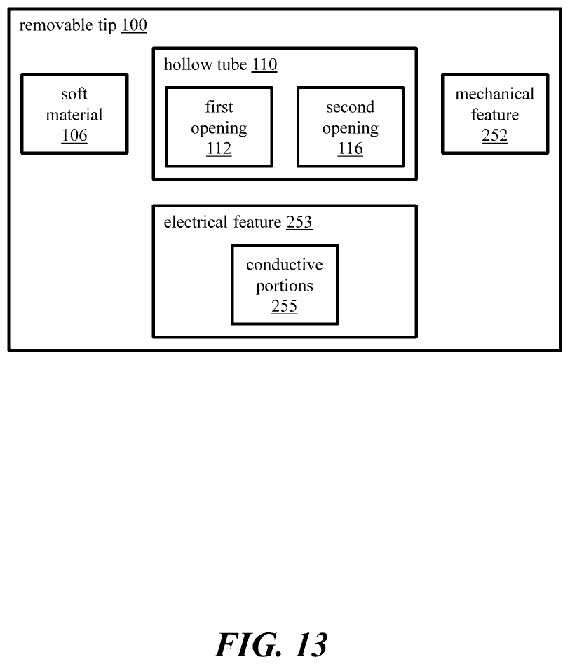

As shown in FIG. 13, the removable tip 100 may include a mechanical feature 252 and/or an electrical feature 253 configured to authenticate and/or pair the removable tip 100 when coupling the removable tip 100 to the nasal cleaning device 102. The electrical feature 253 may include a conductive portion 255 designed to close a circuit within the nasal cleaning device 102 and/or sensors for authenticating surface features on the removable tip 100, as examples. An RFID chip may be embedded within the removable tip 100 and configured to communicate with a reader included in the nasal cleaning device 102 to confirm authenticity. In certain embodiments, a smart chip may be used to store usage data or expiration information. The nasal cleaning device 102 may receive the usage data upon attachment of the removable tip 100 and determine whether the removable tip 100 is suitable for use.

The mechanical feature 252 for authenticating and/or pairing the removable tip 100 may include unique geometric patterns or alignment notches on the removable tip 100 that correspond to complementary structures of the nasal cleaning device 102, thereby ensuring a secure and proper fit. The mechanical feature 252 may also serve as a physical key system, where only a removable tip 100 with the correct mechanical configuration and coding may be attached to the nasal cleaning device 102. Both the electrical feature 253 and the mechanical feature 252 may militate against the use of an incompatible or counterfeit removable tip 100.

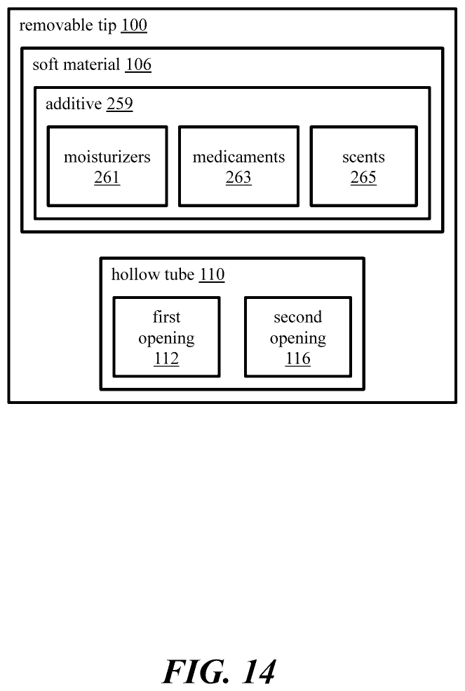

With reference to FIG. 14, the removable tip 100 may include one or more additives 259 providing one or more therapeutic effects. The additive 259 may include a moisturizer 261, a medicament 263, a scent 265, or any combination thereof. In certain embodiments, the applicator body 104 may be impregnated with saline solution 122 for moisturizing the nasal cavity upon application. Examples of additives 259 that may be included for a therapeutic effect further include aloe vera or glycerin for moisturizing, soothing, and hydrating the nasal cavity, menthol or eucalyptus for cooling and refreshing the nasal cavity, and saline solution 122 with xylitol for inhibiting growth of bacteria. Medicaments 263 may include decongestants for reducing swelling and congestion, antihistamines for relieving symptoms relating to allergies, and corticosteroids for treating inflammation. Scents 265 may also be included to enhance the nasal cleaning. Any unique combination of additives 259 may be used in any desired formulations to address specific user needs and preferences, further personalizing each nasal cleaning. One skilled in the art may select suitable additives 259 within the scope of the present disclosure.

As shown in FIGS. 1-3 and 15, the removable tip 100 may be coupled to the nasal cleaning device 102. The nasal cleaning device 102 may include a housing 254, a head assembly 256 including a tip driver 258 configured to vibrate, rotate, or otherwise move the removable tip 100, a basin 260, and a fluid tank (not shown). The fluid tank may include a clean reservoir 257 and a waste reservoir (not shown).

The bottom end 118 of the hollow tube 110 may be configured to be removably coupled to the head assembly 256 of the nasal cleaning device 102. The hollow tube 110 may be coupled to the head assembly 256 using any suitable coupling means such as a friction fit, a screw or threaded fit, a snap fit, an interlocking mechanism, or a press fit, as examples. It should be appreciated that any coupling means capable of allowing a user to easily install and remove the removable tip 100 from the nasal cleaning device 102 while also forming a secure connection between the removable tip 100 to the nasal cleaning device 102 during use may be employed. It should be further appreciated that the coupling means may form a secure enough connection to withstand a vibration, rotation, or any other desired movement of the removable tip 100 from the tip driver 258 during use.

In certain embodiments, a friction fit or interference mechanism may be employed to secure the hollow tube 110 to the tip driver 258 of the nasal cleaning device 102. The outer wall diameter 248 of the hollow tube 110 may be substantially the same as a bore 262 formed in the tip driver 258 of the nasal cleaning device 102. As such, a secure connection may be formed when the bottom end 118 of the hollow tube 110 is forced or otherwise inserted into the bore 262 of the tip driver 258. Any suitable outer wall diameter 248 configured to form a secure friction fit with the bore 262 of the tip driver 258 may be used. Likewise, any suitable length of overlap 264 between the hollow tube 110 and the bore 262 of the tip driver 258 configured to allow for a secure connection may be utilized. In certain embodiments, the tip driver 258 may be fabricated using a thermoplastic elastomer such as santoprene to securely form a seal around the hollow tube 110 upon insertion, thereby forming a secure connection. Other suitable materials for the tip driver 258 may include plastic and rubber, as examples.

The tip driver 258 may include a shelf component 266 configured to secure the hollow tube 110 in a predetermined location 268 with respect to the nasal cleaning device 102. In certain embodiments, the shelf component 266 may be integral with or attached to an inner wall 270 of the tip driver 258. In certain embodiments, the shelf component 266 may be formed by or more sections 271 of the inner wall 270 of the tip driver 258 extending inwardly into a channel 272 of the tip driver 258, thereby preventing insertion of the hollow tube 110 beyond the predetermined location 268. The one or more sections 271 of the inner wall 270 extending inwardly into the channel 272 of the tip driver 258 may be configured to alert the user that the hollow tube 110 has reached the predetermined location 268. The predetermined location 268 may be any location configured to allow a secure connection between the hollow tube 110 and the tipi driver 258 that is capable of withstanding vibration, rotation, and any other desired movement during use.

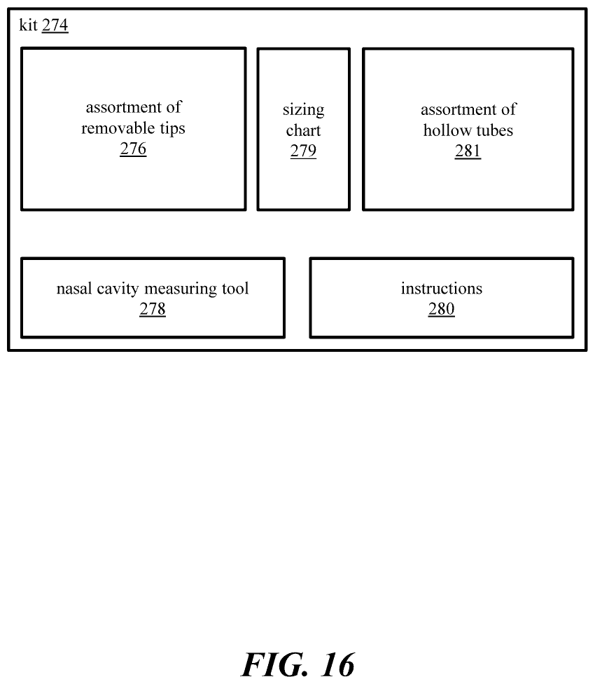

In certain embodiments, as shown in FIG. 16, a kit 274 may include an assortment 276 of removable tips 100, a nasal cavity measuring tool 278, and instructions 280 for selecting a removable tip 100 based on a measured nasal cavity size and user preferences. The kit 274 may include a sizing chart 279 for selecting an appropriate removable tip 100 based on the measured nasal cavity size. Each removable tip 100 in the assortment 276 of removable tips 100 may include an applicator body 104 configured to be applied to a nasal cavity of a user and a hollow tube 110 at least partially disposed in the applicator body 104. The assortment 276 of removable tips 100 may include a plurality of applicator bodies 104 having different sizes, shapes, softness levels, thicknesses, and any other features or combination of features, thereby allowing a user to select an applicator body 104 configured to optimize a nasal cleaning based on the nasal cavity size and user preferences. The assortment 276 of removable tips 100 may further include an assortment 281 of hollow tubes 110 having different configurations. It should be appreciated that the kit 274 may include any number of removable tips 100 and hollow tubes 110, as described herein and within the scope of the present disclosure. It should also be appreciated that, in certain embodiments, the kit may include a nasal cleaning device 102.

Examples of other components that may be included in the kit 274 and configured to enhance nasal cleaning include a saline solution 122 starter pack including pre-measured saline packets for ease of use, disinfectant spray or wipes for cleaning the nasal cleaning device 102 before and after use, and a storage case and/or organizer. Additionally, the kit 274 may include a user manual with detailed instructions, illustrations and tips for maintaining proper nasal hygiene. For educational purposes, an anatomical diagram of the nasal passages may be included. The kit 274 may further include information about a mobile application for downloading that may track nasal cleaning, provide the user with reminders and/or alerts, and offer additional hygiene tips. Additional kit components may be selected to provide a comprehensive nasal cleaning solution, as desired.

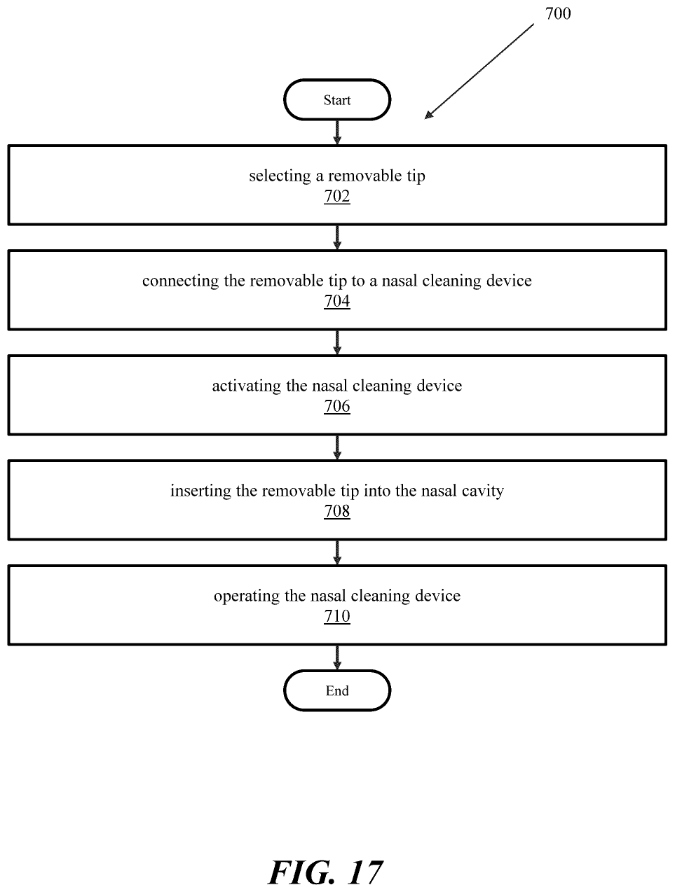

An embodiment of a method 700 of using a removable tip 100 is shown in FIG. 17. In the method 700, a first step 702 may include selecting a removable tip 100 based on the measured nasal cavity size of the user. A second step 704 may include coupling the hollow tube 110 of the removable tip 100 to the nasal cleaning device 102. In certain embodiments, the second step 704 may include coupling the hollow tube 110 of the removable tip 100 to the tip driver 258 of the nasal cleaning device 102. A third step 706 may include activating the nasal cleaning device 102 to deliver saline solution 122 from the clean reservoir 257 to the applicator body 104 of the removable tip 100. In a fourth step 708, the applicator body 104 of the removable tip 100 may be inserted into the nasal cavity of the user. A fifth step 710 may include operating the nasal cleaning device 102 to clean the nasal cavity with the applicator body 104.

The method 700 may include steps relating to measuring the nasal cavity of the user and selecting the proper size, shape, and configuration of the removable tip 100 using a nasal cavity measuring tool 278 configured to measure a with and a height of the nasal cavity, a sizing chart, and/or user preferences. The method 700 may further include steps relating to providing a trial fit of various removable tips 100 initially selected to ensure comfort and proper fit before proceeding with the nasal cleaning. Steps including selecting the proper size, shape, and configuration of the removable tip 100 may include an assessment of user preferences relating to various tip sizes, softness levels, textures, applicator body thickness 134, and any other desired features.

In certain embodiments, the method 700 may include steps such as selecting a removable tip 100 with a desired softness or compressibility level based on user preferences after determining the proper size of the removable tip 100. Steps relating to selecting the proper size, shape, and configuration of the removable tip 100 may include considering the age of the user and the typical nasal cavity size ranges for that age or age group. The method 700 may include following instructions relating to measuring the nasal cavity to enable the user to select the proper size of the removable tip 100. A step of selecting the proper size of the removable tip 100 may include choosing a removable tip 100 with a diameter that is slightly smaller than the measured nasal cavity dimensions to ensure a comfortable but effective fit. Additionally, a step of selecting a removable tip 100 having a desired length of the applicator body 104 may ensure a comfortable and effective fit. It should be appreciated that any number of steps relating to selecting the applicator body 104 and/or the hollow tube 110, establishing secure connections, saturating the applicator body 104, flushing the nasal cavity, operating the nasal cleaning device 102, and cleaning and/or storing the nasal cleaning device 102, as examples, may be included in the method 700. Steps may be repeated or omitted. The order of the steps may be changed, as determined by one of skill in the art.

Advantageously, the removable tip 100 and the associated nasal cleaning device 102, kit 274, and method 700 of the present disclosure may provide enhanced nasal cleaning. Each user may select a removable tip 100 that may be personalized based on nasal cavity size and/or user preferences and needs, as examples. More specifically, varied shapes, sizes, materials, softness and/or compressibility levels, thicknesses, and other desirable features of the removable tip 100, as well as variations in flow patterns and the nasal flush pressure may allow the user to select a removable tip 100 that is configured to provide a comfortable and effective nasal cleaning in line with user anatomy, user preferences, and user needs.

The secure connection between the applicator body 104 and the hollow tube 110, as well as the secure connection between the hollow tube 110 and the tip driver 258, may further enhance a user experience and the nasal cleaning. The removable tip 100 may be easy to install and remove from the nasal cleaning device 102. Additionally, the predetermined location 268 of the hollow tube 110 may be easy to locate using the shelf component 266 and may alert the user when the removable tip 100 is securely attached to the nasal cleaning device 102, as intended. The secure connection between the hollow tube 110 and the tip driver 258 may further enhance the nasal cleaning by allowing to removable tip 100 to vibrate, rotate, and otherwise move effectively and in accordance with the tip driver 258. Likewise, the secure connection between the applicator body 104 and the hollow tube 110 may allow for movement and saturation of the applicator body 104 without impacting the integrity of the removable tip 100. One or more additives 259 may offer additional benefits such as moisturizing, medication delivery, and desirable scents, thereby contributing to an enhanced nasal cleaning. The removable tips 100 having features as described herein and within the scope of the present disclosure may be configured to enhance, personalize, and simplify nasal cleaning.

### EXAMPLES

Example embodiments of the present technology are provided with reference to the several figures enclosed herewith.

In one example, a user suffering from seasonal allergies may select a removable tip 100 having a substantially spherical predetermined shape 108, as shown in FIGS. 8A-8E, and one of a soft or medium softness level configured for gentle application. The user may attach the removable tip 100 to the nasal cleaning device 102, activate the nasal cleaning device 102, and saturate the soft material 106 of the applicator body 104 with saline solution 122. The user may gently insert the removable tip 100 into the nasal cavity. The substantially spherical form of the applicator body 104 along with the softness level may ensure a comfortable cleaning of sensitive areas in the nasal cavity and provide relief from allergens by washing out pollen and dust.

In another example, a pediatric user may require a nasal cleaning due to a respiratory illness. A caregiver may select a removable tip 100 having a small size suitable for a smaller, narrower nasal cavity. The caregiver may couple the removable tip 100 to the nasal cleaning device 102 and activate the nasal cleaning device 102 to deliver saline solution 122 to the soft material 106 of the applicator body 104. The caregiver may insert the removable tip 100 into the nasal cavity of the of the child and carefully clean the nasal cavity, providing a comfortable and effective nasal cleaning without causing distress.

In yet another example, an adult user may have user preferences in line with a more thorough cleaning. As such, the user may select a removable tip 100 with a frustoconical form, as shown in FIGS. 9A-9E, configured to facilitate insertion and maximize contact with the nasal passage walls in the nasal cavity. Further, the selected removable tip 100 may include a hollow tube 110 having apertures 126 and an applicator body thickness 134 configured for an increased nasal flush pressure to further enhance the nasal cleaning. The applicator body 104 may also have a firm softness level. The user may couple the removable tip 100 to the nasal cleaning device 102, insert the nasal cleaning device 102 into the nasal cavity of the user, and activate the nasal cleaning device 102 to deliver the saline solution 122 and dislodge and remove mucus and/or debris using the vibrations, rotations, or other movements of the removable tip 100.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well- known technologies are not described in detail. Equivalent changes, modifications and variations of some embodiments, materials, compositions and methods can be made within the scope of the present technology, with substantially similar results.

What is claimed is:

1. A removable tip for a nasal cleaning device, comprising:
an applicator body having a predetermined shape formed from a compressible material and configured to be applied to a nasal cavity of a user; and
a hollow tube partially disposed in the applicator body, the hollow tube having a first opening at a top end disposed in the applicator body and a second opening at a bottom end,
wherein the bottom end is configured to be removably secured to the nasal cleaning device, selectively placing the hollow tube in fluid communication with a delivery tube configured to deliver a volume of saline solution from a clean reservoir of the nasal cleaning device to the hollow tube and the applicator body of the removable tip.

2. The removable tip of claim 1, wherein the hollow tube has a hollow tube sidewall and wherein a plurality of apertures is disposed in the hollow tube sidewall and configured to release the volume of saline solution directly onto the applicator body.

3. The removable tip of claim 2, wherein the plurality of apertures disposed in the hollow tube sidewall is positioned along a length of the hollow tube that is disposed within the applicator body.

4. The removable tip of claim 1, wherein the applicator body has a predetermined shape selected from a group consisting of a cylindrical shape, a substantially spherical form, and a frustoconical shape, and wherein the predetermined shape is configured to facilitate insertion and removal of the applicator body from the nasal cavity and maximize contact with the nasal cavity.

5. The removable tip of claim 1, wherein the applicator body is removably coupled to the hollow tube using a friction fit.

6. The removable tip of claim 5, wherein an outer wall diameter of the hollow tube is substantially the same as a bore formed in the applicator body and a secure connection is formed when the top end of the hollow tube is inserted into the bore of the applicator body.

7. The removable tip of claim 1, wherein the compressible material of the applicator body includes an additive selected from the group consisting of moisturizers, medicaments, and scents.

8. The removable tip of claim 1, wherein the applicator body includes one of a mechanical feature and an electrical feature configured to authenticate the removable tip with respect to the nasal cleaning device.

9. The removable tip of claim 1, wherein a sidewall portion thickness of the applicator body and a top portion thickness of the applicator body are substantially the same.

10. The removable tip of claim 1, wherein of a first transition area between a sidewall portion and a top portion of the applicator body and a second transition area between the sidewall portion and a bottom portion of the applicator body each include an outer surface having a radius of curvature between 1.0 mm and 2.5 mm.

11. A nasal cleaning device, comprising:
a housing;
a head assembly coupled to the housing, the head assembly having a tip driver and a basin;
a clean reservoir disposed in the housing;
a delivery tube in fluid communication with the clean reservoir; and
a removable tip having
an applicator body having a predetermined shape formed from a compressible material and configured to be applied to a nasal cavity of a user; and
a hollow tube partially disposed in the applicator body, the hollow tube having a first opening at a top end disposed in the applicator body and a second opening at a bottom end,
wherein the bottom end of the hollow tube is configured to be removably secured to the head assembly of the nasal cleaning device, selectively placing the hollow tube in fluid communication with the delivery tube, the delivery tube configured to deliver a volume of saline solution from the clean reservoir of the nasal cleaning device to the hollow tube and the applicator body of the removable tip.

12. The nasal cleaning device of claim 11, wherein the bottom end of the hollow tube is removably secured to the tip driver of the head assembly using a friction fit.

13. The nasal cleaning device of claim 12, wherein an outer wall diameter of the hollow tube is substantially the same as a bore formed in the tip driver and a secure connection is formed when the bottom end of the hollow tube is inserted into the bore of the tip driver.

14. The nasal cleaning device of claim 12, wherein a shelf component disposed in an inner wall of the tip driver is configured to secure the hollow tube in a predetermined location with respect to the nasal cleaning device.

15. The nasal cleaning device of claim 11, wherein the applicator body is removably secured to the hollow tube using a friction fit.

16. A kit for a nasal cleaning device, comprising:
an assortment of removable tips, each removable tip having
an applicator body having a predetermined shape formed from a compressible material and configured to be applied to a nasal cavity of a user, and
a hollow tube at least partially disposed in the applicator body, the hollow tube having a first opening at a top end disposed in the applicator body and a second opening at a bottom end,
wherein the bottom end is configured to be removably secured to the nasal cleaning device, selectively placing the hollow tube in fluid communication with a delivery tube, the delivery tube configured to deliver a volume of saline solution from a clean reservoir of the nasal cleaning device to the hollow tube and the applicator body of the removable tip,
wherein the assortment of removable tips includes a plurality of sizes and compressible materials to accommodate different nasal cavity dimensions and user preferences;
a nasal cavity measuring tool; and
instructions for selecting a removable tip based on a measured nasal cavity size.

17. The nasal cleaning kit of claim 16, wherein the kit includes a sizing chart for selecting an appropriate removable tip based on the measured nasal cavity size.

18. The nasal cleaning kit of claim 16, wherein the kit includes an assortment of hollow tubes having a plurality of hollow tube configurations.

19. A method of selecting and using a removable tip and using the removable tip for cleaning a nasal cavity, the method comprising steps of:
selecting a removable tip based on a nasal cavity of a user and user preferences, the removable tip having
an applicator body having a predetermined shape formed from a compressible material and configured to be applied to the nasal cavity of a user, and
a hollow tube at least partially disposed in the applicator body, the hollow tube having a first opening at a top end disposed in the applicator body and a second opening at a bottom end,
wherein the bottom end is configured to be removably secured to the nasal cleaning device, selectively placing the hollow tube in fluid communication with the delivery tube, the delivery tube configured to deliver a volume of saline solution from a clean reservoir of the nasal cleaning device to the hollow tube and the applicator body of the removable tip;
coupling the bottom end of the hollow tube to the nasal cleaning device;
activating the nasal cleaning device to deliver the volume of saline solution from the clean reservoir to the applicator body of the removable tip;
inserting the applicator body of the removable tip into the nasal cavity of the user; and
operating the nasal cleaning device to clean the nasal cavity.

20. The method of claim 19, wherein the method includes a step of measuring a nasal cavity size of the user to select a proper size of the removable tip, wherein the step of measuring the nasal cavity size includes using a nasal cavity measuring tool configured to measure a width and a height of a nasal cavity.
